(19) United States
(12) Patent Application Publication
(10) Pub. No.: US 2020/0219632 A1
(43) Pub. Date: Jul. 9, 2020

(54) FABRIC FOR SHIELDING A WEARER FROM RADIATION AND GARMENT THEREOF

(71) Applicant: Salini NAIDU, Winterville, NC (US)

(72) Inventor: Salini NAIDU, Winterville, NC (US)

(21) Appl. No.: 16/239,555

(22) Filed: Jan. 4, 2019

(51) Int. Cl. G21F 3/02; A61B 6/10

(52) U.S. Cl. CPC G21F 3/02; A41B 9/12; A61B 6/107

(57) ABSTRACT

The present disclosure provides a fabric for shielding a wearer from radiation. The fabric includes a first layer having a first surface and a second surface. An anti-radiation layer is attached to the second surface of the first layer. The anti-radiation layer is made of a metallic material for shielding the wearer of the fabric from the radiation. The fabric can be shaped to a garment, for providing protection to the wearer, particularly while using electronic gadget.

### TECHNICAL FIELD

The present disclosure relates generally to protective fabrics and, more particularly to, garments for shielding a wearer from radiations emanating during use of electronic gadgets.

### BACKGROUND

Electromagnetic radiation, either ionized or non-ionized, is a naturally occurring phenomenon of electromagnetism. Some of these radiations are characterized to be harmful due to the inherent damage caused to the biological tissues upon exposure. The effects of electromagnetic radiation upon the biological tissues are typically, influenced by the power and frequency of a source of the radiation.

In recent past, electronic gadgets such as, but not limited to, cellular phones, tablets, laptops and the like, have become an integral part of our lives due to their usage and user-friendly characteristics. These electronic gadgets emit radiations in microwave range during use. Although there is no evidence of harm caused due to the use of the electronic gadgets, prolonged exposure of these radiations may inherently lead to severe consequences such as, but not limited to, leukemia, lymphoma, cancer and other complications. These situations are particularly applicable for children/young adults as their bodily systems, such as but not limited to the nervous system, bone marrow, reproductory organs and the like, are still under development. Therefore, children/young adults are typically more vulnerable to factors that may cause severe health consequences. Additionally, the children can now access electronic gadgets readily, which over time, accumulates the exposure to the radiations, which is undesirable.

Therefore, there is a need for techniques which can overcome one or more limitations stated above in addition to providing other technical advantages.

### SUMMARY

Various embodiments of the present disclosure provide a fabric for shielding a wearer from radiation. The fabric includes a first layer having a first surface and a second surface. An anti-radiation layer is attached to the second surface of the first layer, where the anti-radiation layer at least partially includes a metallic material for shielding the wearer of the fabric from the radiation.

In an embodiment of the present disclosure, a garment for shielding the wearer from the radiation is disclosed. The garment includes the fabric adapted to be worn by the wearer. The fabric includes the first layer having the first surface and the second surface. The anti-radiation layer is attached to the second surface of the first layer. The anti-radiation layer is at least partially made of a metallic material for shielding the wearer. A closure mechanism is configured on the garment for securing the garment on the wearer.

In an embodiment of the present disclosure, the garment for shielding the wearer from the radiation is disclosed. The garment includes the fabric adapted to be worn by the wearer. The fabric includes the first layer having the first surface and the second surface. The first surface is an outer surface of the garment and the second surface is the surface oriented towards skin of the wearer upon securing the garment on the wearer. The anti-radiation layer is attached to the second surface of the first layer and is made of a silver material for shielding the wearer of the fabric from the radiation. The anti-radiation layer is attached to the first layer at least in regions, where the first layer encompasses reproductive organs and Red Blood Corpuscles developmental regions of the wearer. A second layer is attached to the anti-radiation layer and is configured to contact the skin of the wearer for comfort while wearing the garment. The closure mechanism is configured on the garment for securing the garment on the wearer.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or a tool and instrumentalities disclosed herein. Moreover, those in the art will understand that the drawings are not to scale. Wherever possible, like elements have been indicated by identical numbers:

FIG. 1 is a planar view of a fabric configured to shield a wearer from radiation, in accordance with an example embodiment of the present disclosure;

FIG. 2 is a sectional view of the fabric of FIG. 1 illustrating various layers of the fabric, in accordance with an example embodiment of the present disclosure;

FIG. 3 is a planar view of the fabric of FIG. 1, in accordance with another example embodiment of the present disclosure;

FIG. 4 is a sectional view of the fabric of FIG. 3 illustrating various layers of the fabric, in accordance with an example embodiment of the present disclosure;

FIG. 5 is a planar view of the fabric configured with a closure mechanism for securing the fabric on the wearer, illustrating various layers of the fabric, in accordance with an example embodiment of the present disclosure;

FIG. 6A is a front view of a garment manufactured from the fabric of FIG. 1, in accordance with an example embodiment of the present disclosure;

FIG. 6B is a rear view of the garment of FIG. 6A, in accordance with an example embodiment of the present disclosure;

FIG. 7A is a front view of the garment manufactured from the fabric of FIG. 3, in accordance with an example embodiment of the present disclosure;

FIG. 7B is a rear view of the garment of FIG. 7A, in accordance with an example embodiment of the present disclosure;

FIG. 8A is a front view of the garment manufactured from the fabric of FIG. 3, in accordance with another example embodiment of the present disclosure;

FIG. 8B is a rear view of the garment of FIG. 8A, in accordance with an example embodiment of the present disclosure;

FIG. 9A is a front view of the garment manufactured from the fabric of FIG. 3, in accordance with another example embodiment of the present disclosure;

FIG. 9B is a rear view of the garment of FIG. 9A, in accordance with an example embodiment of the present disclosure;

FIG. 10A is a front view of the garment manufactured from the fabric of FIG. 3, in accordance with another example embodiment of the present disclosure;

FIG. 10B is a rear view of the garment of FIG. 10A, in accordance with an example embodiment of the present disclosure;

FIG. 11A is a front view of a garment manufactured from the fabric of FIG. 1, in accordance with an example embodiment of the present disclosure;

FIG. 11B is a rear view of the garment of FIG. 11A, in accordance with an example embodiment of the present disclosure;

FIG. 12A is a front view of a garment manufactured from the fabric of FIG. 3, in accordance with an example embodiment of the present disclosure;

FIG. 12B is a rear view of the garment of FIG. 12A, in accordance with an example embodiment of the present disclosure;

FIG. 13 is a front view of the garments of FIGS. 11A-11B and 12A-12B secured to the wearer, in accordance with an example embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearance of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### Overview

Various embodiments of the present disclosure provide a fabric for shielding a wearer from radiation. The fabric includes an anti-shielding material configured to block the radiation, particularly the radiation emanating from the use of electronic gadgets. The present disclosure also provides a garment manufactured from the fabric for shielding the wearer from the radiation, while wearing the garment.

The fabric includes a first layer having a first surface to be an outer surface and a second surface oriented towards the skin of the wearer when secured to the wearer. The first layer is made of a non-metallic material selected from at least one of a cotton material, a polyester material or any material normally used in wearable clothes. The anti-radiation layer which is at least partially made of a metallic material such as but not limited to a silver material is attached to the second surface of the first layer. The anti-radiation layer is configured to shield the wearer from the radiation, particularly from the radiations emanating from the electronic gadgets during use. In some embodiments, the anti-radiation layer may be attached to the first layer at least in regions, where the first layer encompasses reproductive organs and Red Blood Corpuscles (RBC) developmental regions of the wearer. This configuration ensures that the sensitive or most vulnerable regions of the wearer are protected from the radiation during use of the electronic gadgets. A closure mechanism is configured on the fabric, for securing the fabric on the wearer. The closure mechanism may be selected from one of a button mechanism, a strap mechanism, a zipper mechanism, and a snap-fit mechanism. Further, a second layer may be attached to the anti-radiation layer such that, the second layer contacts the skin of the wearer of the fabric for providing comfort.

The present disclosure also provides a garment made of the fabric described above. The garment may be one of a shirt, a trouser, a vest, a brief, a suit and the like. The garment ensures that the user is shielded from the radiation during use of the electronic gadgets.

In an embodiment, the term ‘garment’ may be any piece of fabric worn by the wearer. The ‘garment’ provides the necessary protection to the skin of the wearer from the environmental factors such as but not limited to infectious materials, toxic materials, climatic factors and the like. The configuration of the ‘garment’ is not limited to the embodiments described in the disclosure and may be configured to any shape or size as per feasibility and requirement.

In an embodiment, the term ‘wearer’ may be a person or animal or any other living organism capable of wearing the fabric or the garment.

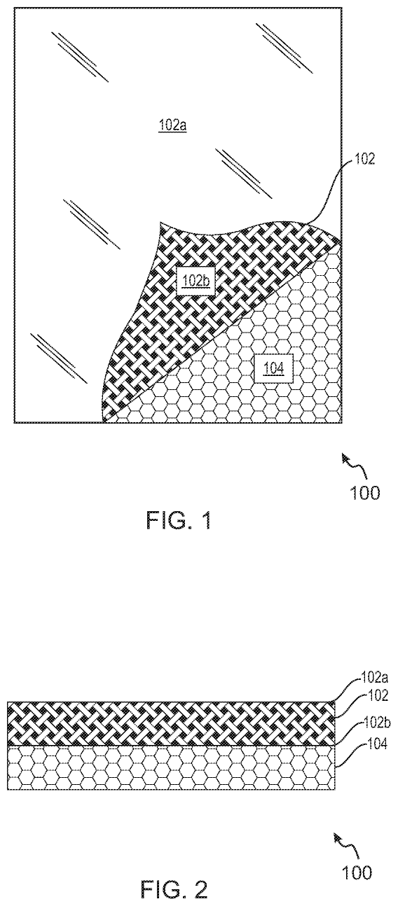

Various embodiments of fabric and garment made thereof for shielding a wearer from radiation are explained herein below, with reference to FIG. 1 to FIG. 13.

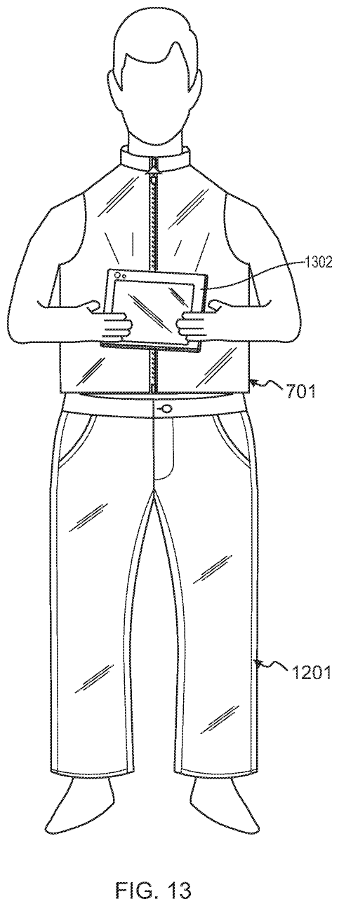

FIG. 1 in one exemplary embodiment of the present disclosure illustrates a planar view of a fabric 100. The fabric 100 is implemented as a wearable material for shielding the wearer from radiations, which can be ionized radiation or non-ionized radiation. The fabric 100 is configured to shield the wearer from the radiations, particularly the radiations emanating during use of electronic gadget 1302 (for e.g. as shown in FIG. 13). This configuration ensures that the wearer is exposed to minimal or no radiations during use of the electronic gadget 1302. Additionally, the fabric 100 also ensures comfort to the wearer while wearing the fabric 100. The fabric 100 may also be tailored to specific requirements of the wearer, while also shielding the wearer from the radiations, thereby ensuring versatility. Although the fabric 100 has been described in terms of independently shielding the radiation, it may be contemplated that the fabric 100 may also be attached on an existing garment or clothing, for protection from the radiations and also for comfort. The fabric 100 may be attached on an existing garment by means such as but not limited to strapping, stitching and the like as per feasibility and requirement.

The fabric 100 includes a first layer 102, an anti-radiation layer 104 and optionally a second layer 302.

The first layer 102 is configured to be leaf-like structure joined by fibers of a non-metallic material by conventional techniques such as but not limited to knitting, weaving and the like. Without loss of generality, the non-metallic material is selected from at least one of a cotton material and a polyester material. The composition of at least one of the cotton material and the polyester material may be selected as per feasibility and requirement. Alternatively, the first layer 102 may be a laminated sheet made of the non-metallic material. The first layer 102 is also configured with a predetermined thickness (for e.g. as shown in FIG. 2) based on the use and application requirement. As an example, the first layer 102 may be made of thicker material for protection from cold weather conditions, while a thinner material may be considered for protection from hot weather conditions. The thickness of the first layer 102 may be varied by altering the configuration of the non-metallic fibers or by altering the configuration of joining each fiber. Furthermore, the first layer 102 may be configured with a predetermined color for enhancing the aesthetic appeal of the fabric 100.

The first layer 102 further includes a first surface 102a and a second surface 102b. The first surface 102a is configured to be an outer surface of the fabric 100 when the fabric 100 is worn by the wearer. The first surface 102a may be configured with patterns or decorations or designs which enhances the aesthetic appeal of the fabric 100. The first surface 102a may also be configured with a fur-type texture for protection of the wearer from climatic factors. Alternatively, the first surface 102a can be configured with texture such as but not limited to a smooth texture, rough texture or any other texture as per requirement. The second surface 102b is configured to be a surface oriented towards skin of the wearer when the fabric 100 is worn by the wearer. As the second surface 102b is oriented towards the skin of the wearer, the texture of the second surface 102b is preferably smooth to prevent discomfort to the wearer while wearing the fabric 100. Alternatively, the second surface 102b can be configured with texture such as but not limited to the rough texture or any other texture as per requirement. The second surface 102b may also be configured with a texture which enhances bonding with another fabric, such as the anti-radiation layer 104 or any other surface as per requirement.

The anti-radiation layer 104 is attached to the second surface 102b of the first layer 102. The anti-radiation layer 104 is attached to the second surface 102b by conventional techniques such as but not limited to adhesive bonding, stitching and the like. In an embodiment, the anti-radiation layer 104 stitched onto the second surface 102b may include pipings or collars (not shown in Figures) for masking the stitches, thereby enhancing aesthetic appeal of the fabric 100. The anti-radiation layer 104 is configured to shield the wearer of the fabric 100 from the radiations. The anti-radiation layer 104 can be the leaf-like structure, similar to the configuration of the first layer 102, made of fibers of a metallic material by conventional techniques such as but not limiting to knitting, weaving and the like. The metallic material is selected from at least one of a copper material, a nickel material, and a gold material. In an embodiment, the metallic material is selected such that, the atomic number of the metallic material is on the higher side in a periodic table. In other words, metallic material with larger atomic size is considered for manufacturing the metallic material required for shielding radiation. In a preferred embodiment, the anti-radiation layer 104 is made of a silver material. In other words, the material made of only silver fibers is employed as the anti-radiation layer 104 (i.e. 100% silver material). The silver material is configured with effectiveness of 100% for shielding the radiation and thus use of the silver material as the anti-radiation layer 104 provides maximum shielding efficacy. Additionally, the silver material also acts as an anti-bacterial layer, thereby protecting the wearer from bacteria. In an embodiment, a composite material made of the metallic materials with higher atomic numbers with respect to the periodic table may be employed as the anti-radiation layer 104. In such a scenario, the composition of the metallic material in the composite material may be tailored suitably, as per requirement. In another embodiment, the anti-radiation layer 104 may be made of about 23% of the copper material, about 20% of the nickel material and about 57% of the polyester material to obtain effectiveness of about 88%. In this configuration of the anti-radiation layer 104, about 88% of the total intensity of the radiations are shielded. Further, the anti-radiation layer 104 is configured with a predetermined thickness (for e.g. as shown in FIG. 2) based on the use and the efficacy of the shielding properties. The thickness of the anti-radiation layer 104 may be varied by altering the configuration of the metallic fibers or by altering the configuration of joining the metallic fibers. In an embodiment, the anti-radiation layer 104 with greater thickness is configured with enhanced shielding properties and vice-versa. Furthermore, the anti-radiation layer 104 is a mesh-like structure, which may also provide structural rigidity to the fabric 100 during use, apart from its radiation shielding characteristics. The type of mesh may depend on the knitting or joining of the fibers used for manufacturing the anti-radiation layer 104. In an embodiment, the mesh structure is selected from one of a honeycomb structure, a hexagonal structure or any other structure as per feasibility and requirement.

Further, the anti-radiation layer 104 may be configured with similar dimensions as that of the first layer 102, and hence attached to the entire area of the first layer 102. This configuration ensures that the anti-radiation layer 104 provides protection to the entire region of the wearer encompassed by the fabric 100. In an embodiment, the anti-radiation layer 104 may be configured in selective regions on the second surface 102b of the first layer 102. The selective regions may be the areas encompassed by the fabric 100 when the fabric 100 is secured on the wearer. The selective regions may be at least one of regenerative organs, bone marrow, Red Blood Corpuscles developmental regions of the wearer. Thus, in this configuration, the anti-radiation layer 104 provides shielding to the most vulnerable regions on the wearer's body, without the need for covering the entire second surface with the anti-radiation layer 104. This scenario is cost-effective while providing protection to the vulnerable region on the wearer's body.

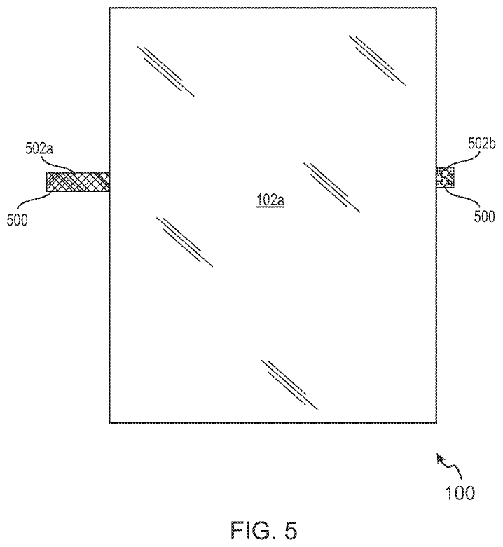

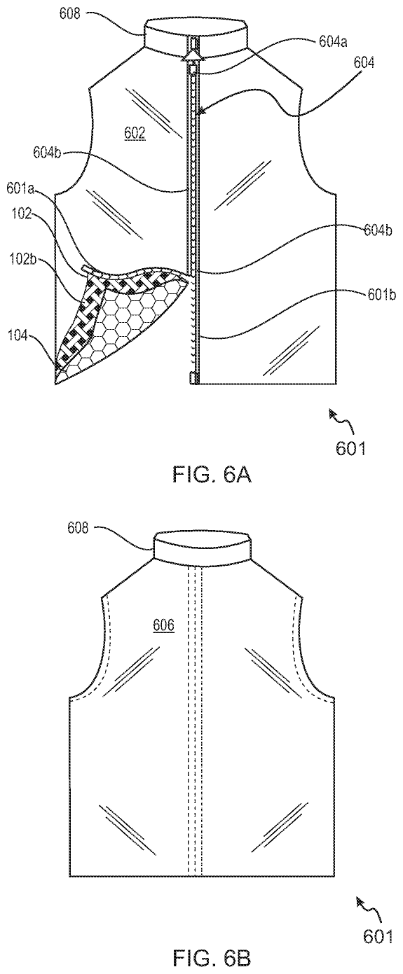

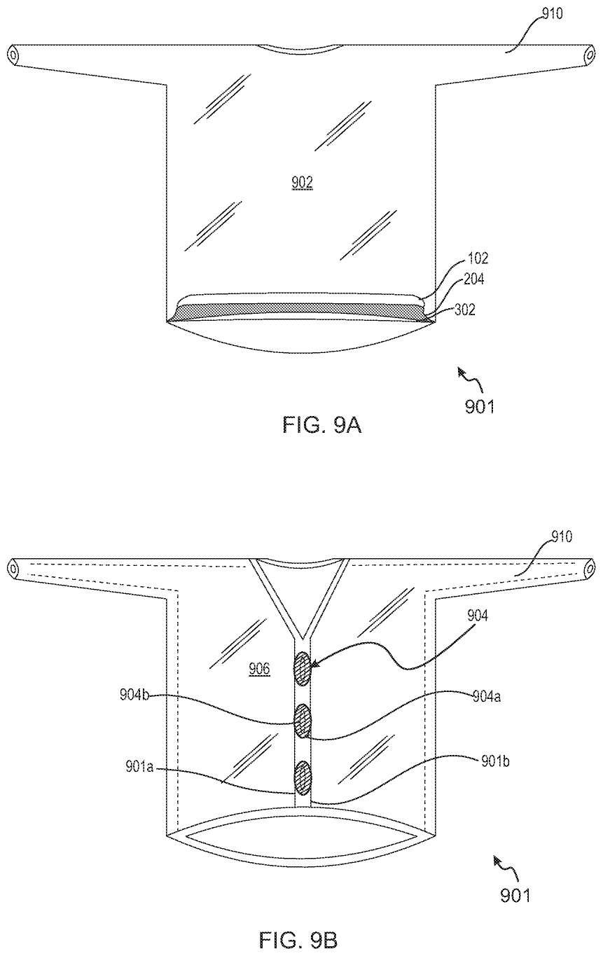

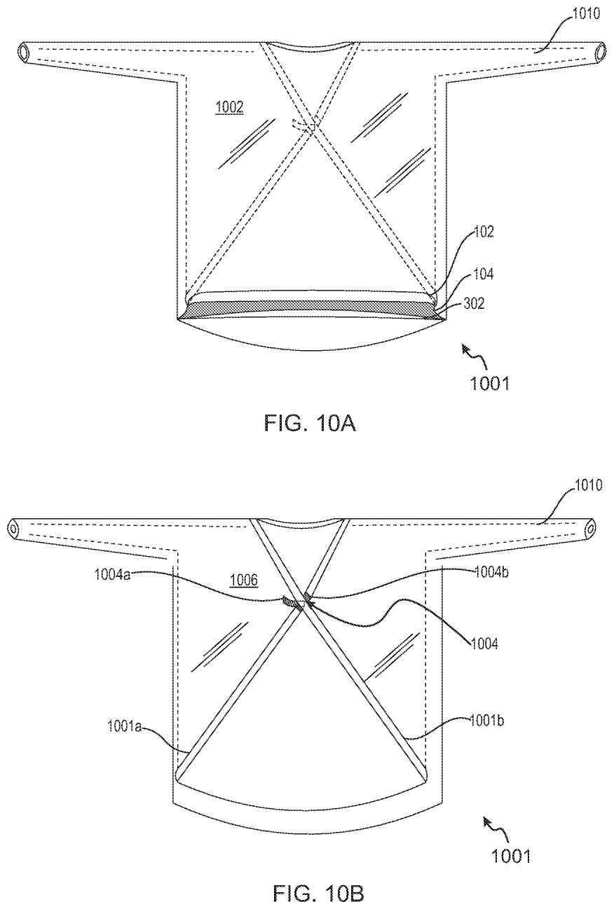

The fabric 100 also includes a closure mechanism 500 (for e.g. as shown in FIG. 5), for securing the fabric 100 on the wearer. The closure mechanism 500 may extend from the first layer 102 or from the anti-radiation layer 104. The closure mechanism 500 may be selected from one of a button mechanism 904 (for e.g. as shown in FIG. 9B), a strap mechanism (not shown in Figures), a Velcro® mechanism 1004 (for e.g. as shown in FIG. 10B), a zipper mechanism 604 (for e.g. as shown in FIG. 6A), a snap-fit mechanism (not shown in Figures) or any other mechanism which serves the purpose of securing the fabric 100 on the wearer. The closure mechanism 500 is positioned appropriately on the fabric 100, based on the type of the closure mechanism 500 employed on the fabric 100 and the type of fitment required for the wearer. In an embodiment, the closure mechanism 500 includes a hook 502a mounted to an end 100a of the fabric 100 and a loop 502b is mounted to another end 100b of the fabric 100. The hook 502a is fastened or attached to the loop 502b, for joining the ends 100a and 100b of the fabric 100.

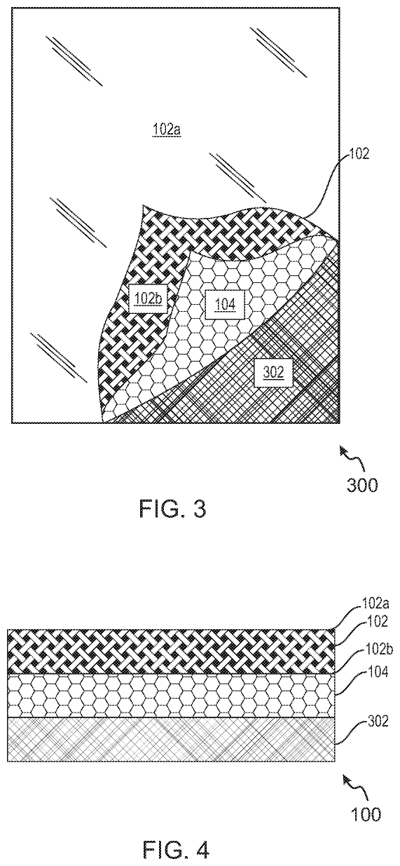

Referring to FIG. 3 in conjunction with FIG. 1, the second layer 302 is optionally attached to the anti-radiation layer 104 of the fabric 100 to form the fabric 300. The second layer 302 is configured to contact the skin of the wearer, while the wearer is wearing the fabric 300. The second layer 302 is configured to contact the wearer's skin for comfort while wearing the fabric 300. The second layer 302 also prevents oxidization of the anti-radiation layer 104, which may occur due to contact of oil secreted from the skin of the wearer, while wearing the fabric 300. Oxidization of the anti-radiation layer 104 may deteriorate the shielding characteristics or properties of the anti-radiation layer 104. Thus, the second layer 302 acts as a protective layer to the anti-radiation layer 104, while also enhancing the comfort of wearing the fabric 300. The second layer 302 is also configured with dimensions similar to either of the anti-radiation layer 104 or the first layer 102. The second layer 302 is at least partially made of the non-metallic material. The non-metallic material is selected from at least one of the cotton material and the polyester material. As the second layer 302 contacts the skin of the wearer, the second layer 302 is preferably made of smoother texture, for enhancing wearer's comfort. In an embodiment, the second layer 302 is made from a blend of the cotton material and the polyester material for a softer feel of the fabric 300. This configuration of the second layer 302 enhances softer feel thereby enhancing comfort, while also improving aesthetic appeal due to the softer feel of the second layer 302. Moreover, the wearer, particularly young adults, may have atopy or allergy or skin rashes at an early age from non-cotton clothing. This configuration of the second layer 302 mitigates the problem of formation of rashes due to the incorporation of the cotton material. In an embodiment, the configuration of the second layer 302 may be similar to the configuration of the first layer 102. In an embodiment, the composition of the non-metallic fibers in the second layer 302 may be selected based on the texture required.

Further, the second layer 302 is configured with a predetermined thickness (for e.g. as shown in FIG. 4) based as per requirement. The thickness of the second layer 302 may be varied by altering the configuration of the non-metallic fibers or by altering the configuration of joining the non-metallic fibers.

FIG. 6A in one exemplary embodiment of the present disclosure illustrates a front view of a garment 601 made of the fabric 100 (i.e. including two layers). The garment 601 is wearable by the wearer for shielding the radiations during use of the electronic gadget 1302 (for e.g. as shown in FIG. 13). The garment 601 illustrated in FIG. 6A is a vest type garment, wherein the ends of the garment are connected by the zipper-type closure mechanism 604. Alternatively, configuration of the garment 601 made of the fabric 100, may be one of a T-shirt (not shown in Figures), a shirt (not shown in Figures), a trouser (not shown in Figures), a brief (not shown in Figures) or a suit (not shown in Figures) or any other garment 601 as per requirement of the wearer, which will be described in subsequent paragraphs.

The vest garment 601 as illustrated in FIG. 6A is made of the fabric 100 as already described above. The ends 601a and 601b of the garment 601 include the zipper-type closure mechanism 604 located at a front portion 602 of the garment 601, for securing the garment 601 on the wearer, while a rear portion 606 (for e.g. as shown in FIG. 6B) of the garment 601 is plain or simple in configuration. The zipper type closure mechanism 604 includes a slider 604a and a toothed rail 604b. The toothed rail 604b is attached to each of the ends 601a and 601b of the garment 601. The slider 604a is mounted on one of the toothed rail 604b, wherein upon engaging the toothed rail 604b of the ends 601a and 601b, the slider 604a is operated, which results in the meshing of the teeth of the toothed rail 604. The meshing of the teeth in the toothed rail 604b results in securing the ends of the garment 601. In this configuration, the slider 604a acts as the hook 502a of the closure mechanism 500, while the toothed rail 604b acts as the loop 502b of the closure mechanism 500. Further, the garment 601 also includes a collar portion 608 configured to receive neck wear (not shown in Figures). In an embodiment, the zipper-type closure mechanism 604 is configured along a central plane of the front portion 602 of the garment 601. Alternatively, the zipper-type closure mechanism 604 may be incorporated at any region or location of the garment 601 as per requirement. This configuration of the vest type garment 601 is configured to provide protection to the torso of the wearer from the radiation.

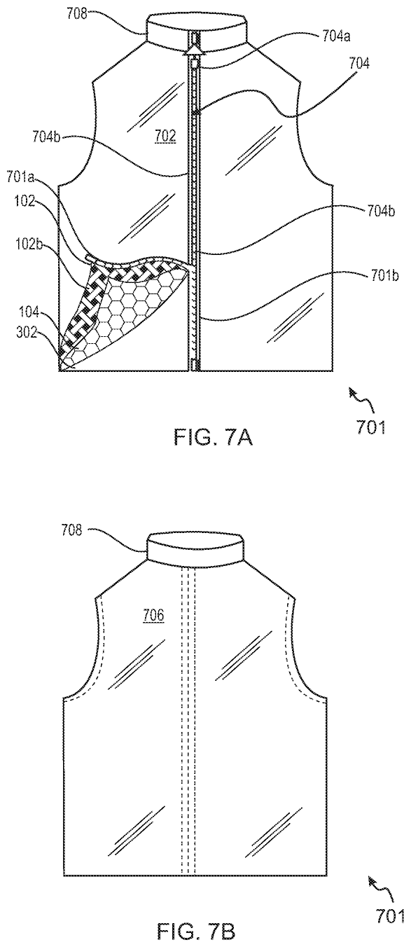

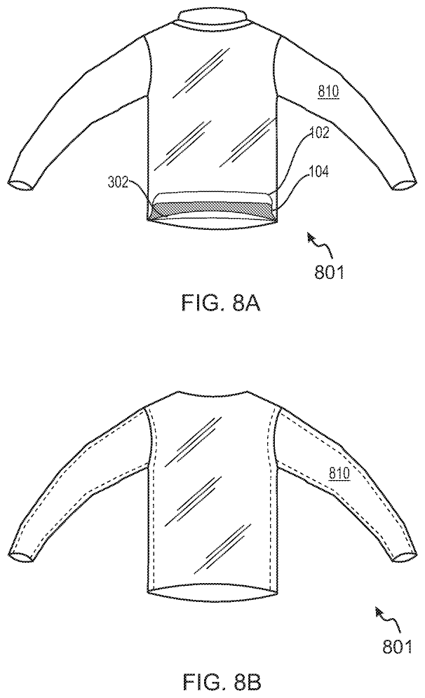

FIG. 7A in one exemplary embodiment of the present disclosure illustrates a front view of a garment 701 made of the fabric 300 (i.e. including three layers). The garment 701 is wearable by the wearer for shielding the radiations during use of the electronic gadget 1302 (for e.g. as shown in FIG. 13). The garment 701 illustrated in FIG. 6 is also the vest type garment, similar to the garment 601, wherein the ends of the garment are connected by a zipper-type closure mechanism 704. Alternatively, configuration of the garment 701 may be one of a T-shirt (for e.g. as shown in FIGS. 8A and 8B), a shirt (for e.g. as shown in FIGS. 9A and 9B), a trouser (not shown in Figures), a brief (not shown in Figures) or a suit (not shown in Figures) or any other garment 601 as per requirement of the wearer, which will be described in subsequent paragraphs.

The vest garment 701 as illustrated in FIG. 7A is made of the fabric 300 as already described above. The ends of the garment 701 include the zipper-type closure mechanism 704 located at a front portion 702 of the garment 601, for securing the garment 701 on the wearer. While a rear portion 706 (for e.g. as shown in FIG. 7B) of the garment 701 is plain or simple in configuration. The zipper-type closure mechanism 704 includes a slider 704a and a toothed rail 704b. The toothed rail 704b is attached to each of the ends 701a and 701b of the garment 701. The slider 704a is mounted on one of the toothed rail 704b, wherein upon engaging the toothed rail 704b of the ends 701a and 701b, the slider 704a is actuated, which results in the meshing of the teeth of the toothed rail 704b. The meshing of the teeth in the toothed rail 704b results in securing the ends of the garment 701. In this configuration, the slider 704a acts as the hook 502a of the closure mechanism 500, while the toothed rail 704b acts as the loop 502b of the closure mechanism 500. Further, the garment 701 also includes a collar portion 708 configured to receive neck wear (not shown in Figures). In an embodiment, the zipper-type closure mechanism 704 is configured along a central plane of the front portion 702 of the garment 701. Alternatively, the zipper-type closure mechanism 704 may be incorporated at any region or location of the garment 701 as per requirement. This configuration of the vest type garment 701 is configured to provide protection to the torso of the wearer from the radiation, while also enhancing comfort while wearing garment 701 due to the second layer 302 of the fabric 300.

Referring to FIGS. 8A and 8B, which illustrates a T-shirt type garment 801 made of the fabric 300. The garment 801 is a cylindrical configuration of the fabric 300, wherein the wearer can directly wear the garment 801 without the need for a closure mechanism 500. The garment 701 exhibits the same characteristics and properties as that of the garment 701. The garment 801 also includes sleeves 810, for protection of the arms of the wearer.

Referring to FIGS. 9A and 9B, which illustrates a jacket type garment 901 made of the fabric 300. The ends 901a and 901b of the garment 901 include a button type closure mechanism 904 located at a rear portion 906 of the garment 901, for securing the garment 901 on the wearer. While a front portion 902 (for e.g. as shown in FIG. 9B) of the garment 901 is plain or simple in configuration. The button type closure mechanism 904 includes buttons 904a attached on one end 901a of the garment 901, while another end 901b includes loops 904b for receiving the buttons 904a. The buttons 904a are fastened within the loops 904b for securing the garment 901 on the wearer. In this configuration of the button type closure mechanism 904, the button 904a acts as the hook 502a of the closure mechanism 500. Also, as the button type closure mechanism 904 is configured on the rear portion 906 of the garment 901, the user is required to fasten the garment 901 to his rear portion. Further, due to the plain configuration of the front portion 902, the front portion of the user is completely protected by the garment 901 from the radiations emanating from the electronic gadget 1302 during use. In an embodiment, the button type closure mechanism 904 is configured along a central plane of the front portion 902 of the garment 901. Alternatively, the button type closure mechanism 904 may be incorporated at any region or location of the garment 901 as per requirement. The garment 901 also includes sleeves 910, for protection of the arms of the wearer.

Referring to FIGS. 10A and 10B, which illustrates a jacket type garment 1001 made of the fabric 300. The garment 1001 includes a Velcro® type closure mechanism 1004 configured on ends 1001a and 1001b, and located along a rear portion 1006 of the garment 1001 (for e.g. as shown in FIG. 10B), for securing the garment 1001 on the wearer. While a front portion 1002 (for e.g. as shown in FIG. 10A) of the garment 1001 is plain or simple in configuration. The Velcro® type closure mechanism 1004 includes a strap member 1004a attached on one end 1001a of the garment 1001, while another end 1001b includes loops 1004b for receiving the strap member 1004a. The strap member 1004a is fastened on the loops 1004b for securing the garment 1001 on the wearer. In this configuration of the Velcro® type closure mechanism 1004, the strap member 1004a acts as the hook 502a of the closure mechanism 500. In an embodiment, the Velcro® type closure mechanism 1004 is configured along a central plane of the front portion 1002 of the garment 1001. Alternatively, the Velcro® type closure mechanism 1004 may be incorporated at any region or location of the garment 1001 as per requirement. The garment 1001 also includes sleeves 1010, for protection of the arms of the wearer. In this configuration, as the Velcro® type closure mechanism 1004 is configured on the rear portion 1006 of the garment 1001, the user is required to fasten the garment 1001 to his rear portion. Also, due to the plain configuration of the front portion 1102, the front portion of the user is completely protected from the radiations emanating from the electronic gadget 1302 during use.

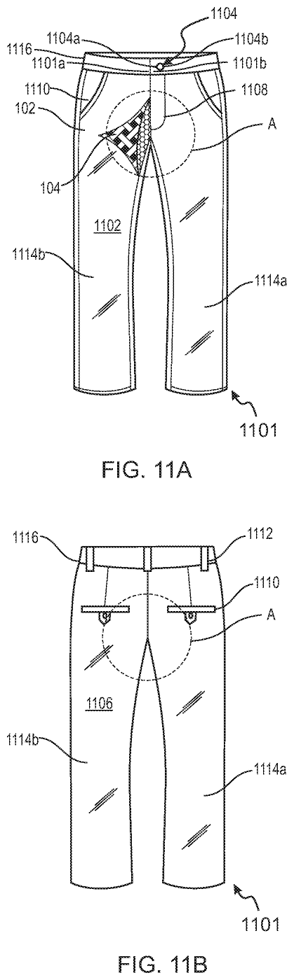

Referring to FIGS. 11A and 11B which illustrates a trouser garment 1101 made of fabric 100. In an embodiment, the garment 1101 may include the fabric 100 in the fly region A (as illustrated by dotted lines) of the garment 1101. This configuration provides protection to the reproductive organs of the user (for e.g. shown as A in FIG. 11A). The ends 1101a and 1101b of the garment 1101 include a button type closure mechanism 1104 located at a front portion 1102 of the garment 1101 (for e.g. as shown in FIG. 11A), for securing the garment 1101 on the wearer. While a rear portion 1106 (for e.g. as shown in FIG. 11B) of the garment 1101 is plain or simple in configuration. The button type closure mechanism 1104 includes a button 1104a attached on one end 1101a of the garment 1101, while another end 1101b includes a loop 1104b for receiving the buttons 1104a. The button 1104a is fastened within the loop 1104b for securing the garment 1101 on the wearer. In this configuration of the button type closure mechanism 1104, the button 1104a acts as the hook 502a of the closure mechanism 500. The garment 1101 includes a waist band 1116 proportionate to the waist of the user. Leg panels 1114a and 1114b extend from the waist band 1116 and include an opening at their free ends for enabling insertion of legs of the user into the leg panels 1114a and 1114b for wearing the garment 1101 (for e.g. as shown in FIG. 13). In an embodiment, the leg panels 1114a and 1114b may be cylindrical structures which may defined based on the styling of the garment 1101. In an embodiment, the leg panels 1114a and 1114b may be configured to be a bell bottom configuration, a tapered configuration and the like as per feasibility and requirement. The garment 1101 also include a fly 1108 positioned below the waist band 1116 and may include a zipper type closure mechanism (not shown in Figures). Further, the rear portion 1106 of the garment 1101 include pockets 1110 for storage of user preferred items. The pockets 1110 may also include the button type closure mechanism 1104, for securely storing the user preferred items within the pockets 1110. Further, the waist band 1116 may also include plurality of belt loops 1112 positioned at predetermined locations along the waist band 1116 for receiving a belt (not shown in Figures). This configuration of the garment 1101 ensures protection for the lower body of the user from the radiations, particularly, the reproductive organs of the user.

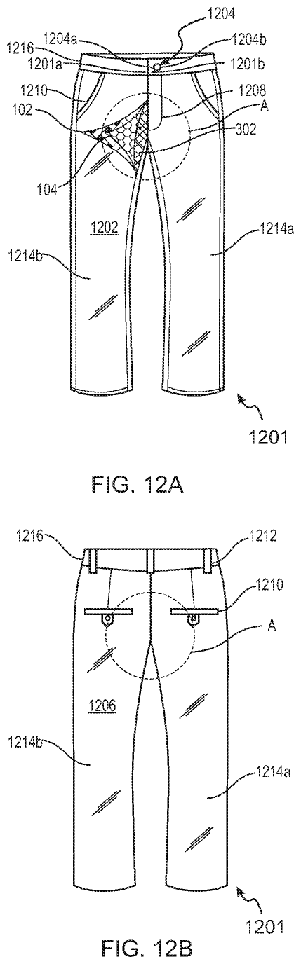

Referring to FIGS. 12A and 12B which illustrate another trouser garment 1201 made of fabric 300. In an embodiment, the garment 1201 may include the fabric 300 in the fly region A (as illustrated by dotted lines) of the garment 1201. This configuration provides protection to the reproductive organs of the user. The ends 1201a and 1201b of the garment 1201 include a button type closure mechanism 1204 located at a front portion 1202 of the garment 1201 (for e.g. as shown in FIG. 12A), for securing the garment 1201 on the wearer. The rear portion 1202 (for e.g. as shown in FIG. 12B) of the garment 1201 is plain or simple in configuration. The button type closure mechanism 1204 includes a button 1204a attached on one end 1201a of the garment 1201, while another end 1201b includes a loop 1204b for receiving the buttons 1204a. The button 1204a is fastened within the loop 1204b for securing the garment 1201 on the wearer. In this configuration of the button type closure mechanism 1204, the button 1204a acts as the hook 502a of the closure mechanism 500. The garment 1201 includes a waist band 1216 proportionate to the waist of the user. Leg panels 1214a and 1214b extend from the waist band 1216 and include an opening at their free ends for enabling insertion of legs of the user into the leg panels 1214a and 1214b for wearing the garment 1201 (for e.g. as shown in FIG. 13). In an embodiment, the leg panels 1214a and 1214b may be cylindrical structures which may defined based on the styling of the garment 1201. In an embodiment, the leg panels 1214a and 1214b may be configured to be a bell bottom configuration, a tapered configuration and the like as per feasibility and requirement. The garment 1201 also includes a fly 1208 positioned below the waist band 1216 and may include a zipper type closure mechanism (not shown in Figures). Further, the rear portion 1206 of the garment 1201 include pockets 1210 for storage of user preferred items. The pockets 1210 may also include the button type closure mechanism 1204, for securely storing the user preferred items within the pockets 1210. Further, the waist band 1116 may also include a plurality of belt loops 1212 positioned at predetermined locations along the waist band 1216 for receiving a belt (not shown in Figures). This configuration of the garment 1201 ensures protection for the lower body of the user from the radiations, particularly, the reproductive organs of the user, while also ensuring user's comfort while wearing the garment 1201.

In an embodiment, the garment such as garments 601,701,801,901,1001, 1101 or 1201, may be configured to scatter the radiation directed towards the wearer during use of the electronic gadget 1302 (for e.g. as shown in FIG. 13), due to an interaction of particles in the radiation with the atoms of the anti-radiation layer 104.

The benefits and advantages described above may relate to one embodiment or may relate to several embodiments. The embodiments are not limited to those that solve any or all of the stated problems or those that have any or all of the stated benefits and advantages.

The above description is given by way of example only and various modifications may be made by those skilled in the art. The above specification, examples, and data provide a complete description of the structure and use of exemplary embodiments. Although various embodiments have been described above with a certain degree of particularity, or with reference to one or more individual embodiments, those skilled in the art could make numerous alterations to the disclosed embodiments without departing from the spirit or scope of this specification.

1. A fabric for shielding a wearer from radiation, the fabric comprising:
a first layer including a first surface and a second surface; and
an anti-radiation layer attached to the second surface of the first layer at one or more selective regions, wherein the anti-radiation layer is made of 23% of a copper material, 20% of a nickel material and 57% of a polyester material for shielding the wearer of the fabric from the radiation.

2. The fabric as claimed in claim 1, further comprising a closure mechanism for securing the fabric on the wearer.

3. The fabric as claimed in claim 2, wherein the closure mechanism is one of: a button mechanism, a strap mechanism, a zipper mechanism and a snap-fit mechanism.

4. The fabric as claimed in claim 1, wherein the first surface is an outer surface of the fabric and the second surface is the surface oriented towards skin of the wearer when the fabric is secured on the wearer.

5. The fabric as claimed in claim 1, wherein the first fabric comprises non-metallic materials, the non-metallic materials including at least one of a cotton material and a polyester material.

6. The fabric as claimed in claim 1, wherein the anti-radiation layer is attached to the first layer at least in regions, where the first layer encompasses reproductive organs and Red Blood Corpuscles developmental regions of the wearer.

7. The fabric as claimed in claim 1, wherein the anti-radiation layer is made of a silver material.

8. (canceled)

9. The fabric as claimed in claim 1, further comprising a second layer attached to the anti-radiation layer, the second layer contacting skin of the wearer for comfort while wearing the fabric.

10. The fabric as claimed in claim 9, wherein the second layer comprises non-metallic materials, the non-metallic materials including at least one of a cotton material and a polyester material.

11. A garment for shielding a wearer from radiation, the garment comprising:
a fabric adapted to be worn by the wearer, the fabric including:
a first layer including a first surface and a second surface; and an anti-radiation layer attached to the second surface of the first layer at one or more selective regions, wherein the anti-radiation layer is made of 23% of a copper material, 20% of a nickel material and 57% of a polyester material for shielding the wearer; and
a closure mechanism configured on the garment for securing the garment on the wearer.

12. The garment as claimed in claim 11, wherein the first layer comprises non-metallic materials, the non-metallic materials including at least one of a cotton material and a polyester material.

13. The garment as claimed in claim 11, wherein the anti-radiation layer is made of a silver material.

14. (canceled)

15. The garment as claimed in claim 11, wherein the anti-radiation layer is attached to the first layer at least in regions, where the first layer encompasses reproductive organs and Red Blood Corpuscles developmental regions of the wearer.

16. The garment as claimed in claim 11, further comprising a second layer attached to the anti-radiation layer, the second layer contacting skin of the wearer for comfort while wearing the garment.

17. The garment as claimed in claim 11, wherein the closure mechanism is one of: a button mechanism, a strap mechanism, a zipper mechanism, and a snap-fit mechanism.

18. The garment as claimed in claim 11, wherein the garment is one of: a shirt, a trouser, a vest, a brief, a jacket, and a suit.

19. A garment for shielding a wearer from radiation, the garment comprising:
a fabric adapted to be worn by the wearer, the fabric comprising:
a first layer including a first surface and a second surface, the first surface being an outer surface of the garment and the second surface being oriented towards skin of the wearer upon securing the garment on the wearer,
an anti-radiation layer attached to the second surface of the first layer, wherein the anti-radiation layer is made of 23% of a copper material, 20% of a nickel material and 57% of a polyester material for shielding the wearer of the fabric from the radiation, wherein the anti-radiation layer is attached to the first layer at one or more selective regions, and wherein the first layer encompasses reproductive organs and Red Blood Corpuscles developmental regions of the wearer; and
a second layer attached to the anti-radiation layer, the second layer contacting skin of the wearer for comfort while wearing the garment; and
a closure mechanism configured on the garment for securing the garment on the wearer.

20. The garment as claimed in claim 19, wherein the radiation shielded by the garment is at least one of ionized radiation or non-ionizing radiation.
